(19) United States
(12) Patent Application Publication
(10) Pub. No.: US 2014/0113388 A1
(43) Pub. Date: Apr. 24, 2014

(54) TREATMENT PLANNING BASED ON POLYPEPTIDE RADIOTOXICITY SERUM MARKERS

(71) Applicants: Katrin Bitter, Aachen (DE); Carolina Ribbing, Aachen (DE)

(72) Inventors: Katrin Bitter, Aachen (DE); Carolina Ribbing, Aachen (DE)

(73) Assignee: KONINKLIJKE PHILIPS N.V., EINDHOVEN (NL)

(21) Appl. No.: 14/125,991

(22) PCT Filed: Jun. 29, 2012
(86) PCT No.: PCT/IB2012/053323

§ 371 Date: Dec. 13, 2013

Related U.S. Application Data
(60) Provisional application No. 61/502,960, filed on Jun. 30, 2011.

(51) Int. Cl. A61N 5/10

(52) U.S. Cl. CPC A61N 5/1038
USPC 436/501; 600/1

(57) ABSTRACT

A method includes at least one of creating or adapting a treatment plan for a patient based on a set of serum polypeptides of the patient that are indicative of a radiotoxicity of the patient at least one of before or after at least one of a plurality of radiotherapy treatments of the treatment plan, wherein the radiotoxicity is induced by radiation exposure from the radiotherapy treatment. A system includes a treatment planning device (108) that facilitates at least one of creating or adapting a treatment plan for a patient based on amounts or concentrations of a set of serum polypeptides of the patient that indicate a high risk of or an early radiotoxicity of the patient to radiation from radiotherapy.

### FIELD OF THE INVENTION

The following generally relates to treatment planning and more particularly to creating and/or adapting a treatment plan for a patient based on a set of polypeptide serum markers of the patient that can be used to predict, early detect, and/or monitor radiotoxicity of the patient induced by radiation from radiotherapy.

### BACKGROUND OF THE INVENTION

Generally, events occurring in the body are molecularly mediated, mostly by proteins. Ongoing physiological or pathological events are represented by the relative cellular abundance of tens of thousands of different proteins along with their chemically modified and cleaved forms. Every cell gives an account of its physiological state in the molecular products it contains and releases. Within molecular diagnostics (MDx), some of the cellular products from this diagnostic information mine are used as disease markers or as pathological fingerprints. The outcome of such tests may be important input for any decision support tool that combines diagnosis and disease prognosis.

Mass spectrometry (MS) is a method for determining molecular mass, involving sample ionization and transfer to the gas phase. By acceleration in an electric field and separation in vacuum, the molecular ions are separated according to their mass-to-charge ratio. During the last decades, MS has proven to be a viable technique for accurate and sensitive analysis of biological species like proteins and peptides. With the introduction of soft ionization techniques, it became possible to transfer these non-volatile, large, and thermally labile molecules into the gas phase without dissociating them.

In matrix-assisted laser desorption ionization (MALDI), the sample is co-crystallized with a UV absorbing aromatic compound which is added to the sample in large excess. Common UV absorbing matrices include a-cyano-4-hydroxy cinnamic acid (CHCA) and 3,5-dimethoxy-4-hydroxy cinnamic acid (sinapinic acid). A pulsed UV laser supplies the energy for ionization and desorption, and the matrix absorbs the UV energy and transfers it to the sample. Typically, a N₂ laser with 337 nm wavelength (3.7 eV) and e.g., 4 ns pulses is used. As comparison, about 13-14 eV is required for one ˜12 kDa (Dalton) molecule to be desorbed and ionized. Using MALDI-MS, molecules with masses exceeding 105 Da can be ionized and analyzed without appreciable fragmentation.

Prior to performing MALDI-MS, complex samples like molecular digests, cell lysates and blood serum have to be pre-fractionated in order to eliminate the suppression of molecular desorption/ionization often observed with complex mixtures (ion suppression), to avoid too heterogeneous sample compositions and to avoid detector overload. Common pre-fractionation methods include liquid chromatography, electrophoresis, isoelectric focusing, desalting, and removal of particles by centrifugation, as well as concentration and dilution. Often, 2D gel electrophoresis is performed; spots of interests are excised from the gel and dissolved for subsequent MALDI-MS analysis. Another common arrangement is liquid chromatography (LC) coupled directly to another type of mass spectrometer with electrospray ionization (ESI-MS), corresponding to a low-resolution mass separation (LC) in series with a high-resolution mass separation (MS).

MALDI was further refined by introduction of a combination with chromatographic sample pre-fractionation in surface-enhanced affinity capture (SEAC), later surface enhanced laser desorption ionization (SELDI), and by covalent binding of matrix to the sample holding plate in an approach called surface-enhanced neat desorption (SEND). In SELDI, the sample is brought into contact with a chromatographic surface which binds a subgroup of the sample molecules. For sample preparation, individual chromatographic chips are accommodated in a special holder (a bio-processor) to achieve a standard microtiter plate format. Unbound molecules are removed by buffer washing, and a MALDI-MS measurement is performed directly off the chromatographic surface. Matrix is either added as a last step before MS measurement, or is already covalently bound to the chip surface. Only little or no fragmentation is observed.

As an example, when using a hydrophobic surface in SELDI, the subgroup of hydrophobic molecules will be fished out of a complex sample. For biomarker discovery, protein expression profiling, and diagnostic purposes, this is useful for investigation or diagnosis of diseases which lead to a change in the expression of hydrophobic peptides. SELDI advantages include that the sample is concentrated directly on a chromatographic surface in a relatively short process with high throughput potential. The chromatographic MS targets can be automatically loaded with a sample, prepared, and analyzed in the MS. Therefore, the method is interesting for diagnostic applications. The SELDI-TOF mass spectrometers have a simple design and are installed in many clinics and clinical chemistry departments of hospitals.

From blood serum, diagnostic mass spectrometric proteomic patterns showing e.g. early cancer or host response to radiation can be obtained. The literature has indicated that such a diagnostic peptide pattern has enabled early diagnosis of ovarial cancer. The approach of a spectral pattern as a diagnostic discriminator represented a new diagnostic paradigm. For the first time, the pattern itself was the discriminator, independent of the identity of the proteins or peptides. The underlying thesis was that pathological changes within an organ are reflected in proteomic patterns in serum. This is plausible because, generally speaking, and as stated in the opening paragraph, every event occurring in our bodies is molecularly mediated, mostly by proteins.

Tumors are often treated with radiotherapy. In radiotherapy, a radiation dose high enough to kill tumor cells is delivered to the tumor, while trying to spare healthy tissue surrounding the tumor and extra sensitive tissue like epithelial linings, rectum, bowel, urethra, bladder and certain nerve bundles. In external beam radiotherapy, there are always portions of healthy tissue that are exposed to and damaged by radiation. In addition, some patients react with severe side-effects, which have a severe influence on the patient's quality of life. By way of non-limiting example, acute and late toxicity of the bowel and the urinary tract are impeding side-effects in radiotherapy of prostate cancer. With this cancer, radiotherapy planning targets the prostate cancer while minimizing dose to the very closely situated bowel and bladder. The frequent and serious side-effects of prostate cancer radiotherapy especially affect the bladder and the bowel. For example, the side-effects include incontinence, bleeding, pain, etc. Other side-effects include impotence. Other cancers in this bodily region treated using radiotherapy include, but are not limited to, bladder, kidney, bowel, rectum, endometrial, cervix, ovarial or vaginal cancer. With all of these, there may be severe side-effects that may influence the patient's quality of life.

To measure health related quality of life among men with prostate cancer, the Expanded Prostate cancer Index Composite (EPIC) was developed. EPIC consists of a questionnaire that is manually filled out by patients at several time points before, during and after radiotherapy. It assesses the disease-specific aspects of prostate cancer and its therapies and comprises the four summary domains: urinary, bowel, sexual and hormonal. Generally, higher EPIC scores are indicative of a better health-related quality of life. EPIC is a valuable tool for standardized assessment of radiotherapy side-effects and how these effects are perceived by the individual patients. However, EPIC can only report subjectively experienced effects. Furthermore, as with all patient-reported questionnaires, EPIC provides no reliable objective measure of side-effects. Because of at least these drawbacks, EPIC is not well suited to assist in individualization of treatment planning.

### SUMMARY OF THE INVENTION

Aspects of the present application address the above-referenced matters, and others.

In one aspect, a method includes at least one of creating or adapting a treatment plan for a patient based on a set of serum polypeptides of the patient that are indicative of a radiotoxicity of the patient at least one of before or after at least one of a plurality of radiotherapy treatments of the treatment plan, wherein the radiotoxicity is induced by radiation exposure from the radiotherapy treatment.

In another aspect, a system includes a treatment planning device (108) that facilitates at least one of creating or adapting a treatment plan for a patient based amounts or concentrations of a set of serum polypeptides of the patient that indicate a high risk of or an early radiotoxicity of the patient to radiation from radiotherapy.

In another aspect, computer readable storage medium is encoded with computer readable instructions, which, when executed by a processor of a computing system, causes the system to: receive information about a polypeptide of a patient that indicates a radiotoxicity of the patient to radiotherapy treatment and create or adapt a treatment plan for the patient based on the received information, wherein the information includes at least a mass of the polypeptide and an intensity peak of the polypeptide.

Still further aspects of the present invention will be appreciated to those of ordinary skill in the art upon reading and understanding the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example system including a therapy treatment planning device.

FIGS. 2-11 shows information about several polypeptide radiotoxicity serum markers.

FIG. 12 illustrates an example method for treatment planning.

### DETAILED DESCRIPTION OF EMBODIMENTS

The following describes an approach for creating and/or adapting a treatment plan for a patient based on serum concentrations/amounts of a predetermined set of polypeptides of the patient that indicate a radiotoxicity of the patient to radiation from radiotherapy.

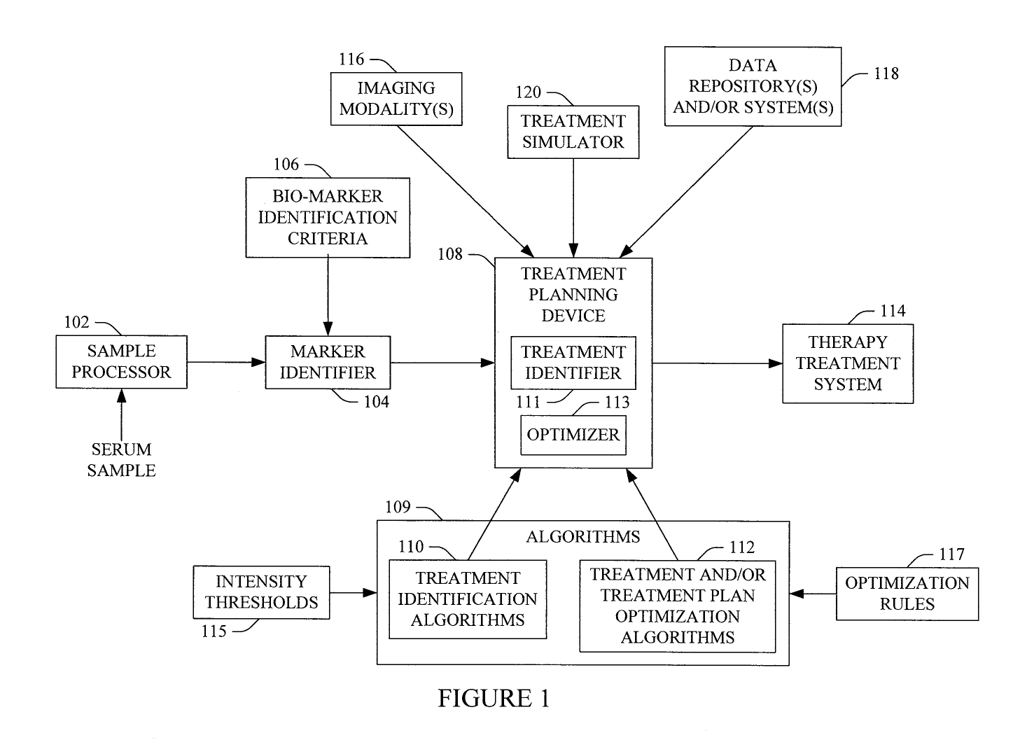

Initially referring to FIG. 1, a sample processor 102 is configured to process serum samples of a patient that include polypeptides, which can be used to predict and/or monitor radiotoxicity of a patient induced by radiation from radiotherapy, and generated a signal indicative thereof. An example of a suitable serum-sample includes blood serum or other serum-sample. An example sample processor 102 is configured to perform mass spectrometry to measure masses and relative amounts of polypeptides in the serum sample and/or the concentrations of the polypeptides in the serum sample.

When predicting radiotoxicity, the serum sample is obtained from the patient prior to radiotherapy, and the prediction can be used to select treatment therapies and create a treatment plan, which may or may not include radiotherapy. For monitoring radiotoxicity, one or more serum samples are obtained respectively during radiotherapy treatment (e.g., after a first, a second, etc. of several scheduled radiotherapy treatments), and the monitored radiotoxicity can be used to adapt the created treatment plan (adaptive re-planning).

A marker identifier 104 is configured to analyze the data generated by the sample processor 102 and identify a sub-set of the polypeptides of the serum sample that correspond to a set of polypeptide radiotoxicity bio-markers of interest. The set of polypeptide radiotoxicity bio-markers of interest are identified based on bio-marker identification criteria 106. In this example, the identification criteria 106 includes polypeptides with masses of 11,668±23 Da, 2,876±6 Da, 6,432±13 Da, 9,125±18 Da, 2,220±4 Da, 9,414±19 Da, and 14,571±29 Da.

It is to be understood that as utilized herein the term “identify,” in the context of the marker identifier 104, refers to identifying bio-markers that have a mass of interest from the criteria 106 from bio-markers that have a mass other than a mass of interest from the criteria 106. Other sets of masses and/or criteria are also contemplated herein. The particular set of criteria 106 can be determined theoretically, empirically, based on previously implemented treatment plans, etc. The bio-marker identifier 104 generates an electronic signal that includes the identified set of polypeptides, along with data such as their masses, peak signal intensity, etc.

Where the serum sample is processed via an immunoassay, marker identifier 104 can be omitted because the assay tests bind to already pre-determined antibodies (i.e., the type of antibodies are on the assay determines which biomarkers are measured).

A treatment planning device 108 is configured to create and/or adapt treatment plans, with or without human interaction, at least based on the signal generated by the bio-marker identifier 104, which includes the identified set of polypeptide radiotoxicity markers along with data such as their masses, peak intensity, etc., and one or more algorithms 109, including treatment identification algorithms 110, optimization algorithms 112, and/or other algorithms. Generally, treatment plan creation includes creating a treatment plan to be implemented and treatment plan adaption includes modifying a treatment plan being implemented. The algorithms 109 can be used with both treatment plan creation and treatment plan adaption.

The illustrated treatment planning device 108 includes a treatment identifier 111 configured to employ the treatment identification algorithms 110 to identify a set of treatments for the plan based on the identified set of polypeptide radiotoxicity markers. Suitable treatments include one or more of external beam radiotherapy, low dose rate (LDR) and/or high dose rate (HDR) brachytherapy, surgery, chemotherapy, particle (e.g., proton) therapy, high intensity focused ultrasound (HIFU), ablation, hormonal therapy, cryotherapy, watchful waiting, and/or other treatments.

The treatment planning device 108 can automatically select and include the identified set of treatments in the plan or recommend the identified set of treatments for the plan to facilitate a user with selecting treatments for the plan. As such, the treatment planning device 108 can be part of or used in connection with a clinical decision support system or a computer aided diagnosis/treatment system.

In one non-limiting embodiment, the identification algorithms 110 compare, for each polypeptide of the identified set of polypeptide radiotoxicity bio-markers, the intensity peak with a corresponding pre-determined intensity threshold value of predetermined intensity thresholds 115. Comparisons at particular radiotherapy time points (e.g., before and/or after one or more radiotherapy treatments) and/or patterns across all or a sub-set of the time points can be used to classify the polypeptide radiotoxicity markers as indicating the patient has higher or lower radiotoxicity based on the thresholds 115. In turn, the treatment identifier 111 can classify a patient as extra radiosensitive or not based on a combination of the polypeptide classifications, and subsequently the treatments in the plan can be personalized for the patient.

The treatment planning device 108 also includes an optimizer 113 configured to employ the optimization algorithms 112 to optimize a treatment (e.g., an external beam radiotherapy treatment) of the plan and/or the treatment plan based on a set of optimization rules 117. The rules 117 may include modifying parameters of one or more of the treatments of the treatment plan. For example, where the set of polypeptide radiotoxicity markers indicate a patient is extra radiosensitive, the rules 117 may indicate that an extra radiation dose boost, which might be beneficial to treating a tumor, should not be performed, extra strict dose limits should be applied for the patient, a change to another treatment of the plan in substitution to the extra radiation dose boost, a modification to a dose distribution contour should be made, etc. As such, individual treatments can be personalized to the patient based on the polypeptide radiosensitivity bio-markers.

The identified set of treatments, the treatments treatment plan, the peak intensity information of the polypeptides, the intensity thresholds 115, the classification of the polypeptides (e.g., as indicating higher or lower radiosensitivity), the classification of the patient (e.g., as having higher or lower radiosensitivity), and/or other information can be visually presented via a display, for example, for confirmation, observation, and/or notification to authorized personnel, printed, stored in computer memory, and/or otherwise processed. This information can be variously formatted such as a table or a graph, as a toxicity index for the patient, and/or otherwise. The data can be colored coded or otherwise visually emphasized or highlighted in order to bring certain information (e.g., the patient is extra radiosensitive) to the user of the treatment planning device 108. The user of the device 108 can utilize all, any of the above-noted and/or other information to create and/or adapt a treatment plan.

A therapy treatment system 114, in the illustrated embodiment, is configured to receive and process the treatment plan from the treatment planning device 108. Examples of suitable therapy treatment systems, include, but are not limited to, an external beam radiation therapy system, a device that facilitates chemotherapy administration, a device that facilitates brachytherapy seed implantation, a particle (e.g., proton) therapy system, a high intensity focused ultrasound (HIFU) system, and/or other treatment system and/or device that facilitates treatment. In one non-limiting instance, the therapy treatment system 114 automatically loads the received treatment plan into the system and/or automatically sets one or more treatment deliver parameters based thereon. In another instance, the therapy treatment system 114 loads the received treatment plan into the system and prompts the user for instructions, which may include accepting the plan or rejecting the plan. In another instance, the therapy treatment system 114 is manually configured by authorized personnel based on the treatment plan.

Other data that can additionally or alternatively be used by the treatment planning device 108 includes, but is not limited to, imaging data from an imaging modality(s) 116, non-imaging data from a repository(s) and/or system 118, a treatment simulation from a treatment simulator 120, existing treatment plans (for the patient and/or other patient(s)) and/or other data.

Suitable imaging modality(s) 116 may include, but is not limited to, computed tomography (CT), positron emission tomography (PET), single photon emission computed tomography (SPECT), magnetic resonance (MR), and/or other imaging data. Functional imaging data can be used to provide tracer uptake information, which may help locate, stage, monitor growth, and monitor response to treatment, and structural imaging data can be used to show morphological changes, such as changes in tissue size, and can be performed weeks after treatment, after the body has had time to respond to the dead cells, in order to determine whether treated tissue has shrunk or grown.

The data from the data repository(s) and/or system(s) 118 may include, but is not limited to, patient history (including medical and/or non-medical), laboratory results, medical and/or non-medical history of other patients, models, pathology, histology, pharmaceutical prescribed and taken by the patient, tumor grading, diagnoses, and/or other data that can be used to predict and/or monitor the dose to be imparted and/or imparted to a target and other regions of the subject by the therapy treatment device 108 and/or other system.

The treatment simulator 120 can be used to simulate the response and/or development of treated and/or untreated structures to be treated in the patient and predict how one or more of the different structures are likely to respond and/or develop with and/or without treatment. The simulator 120 generates an output signal indicative of the simulation, the simulation results, and/or the prediction.

It is to be appreciated that the bio-marker identifier 104 and/or the treatment planning device 108 include one or more processors that execute one or more computable executable instructions stored on computer readable medium such as physical memory to implement the functionality described herein and/or other functionality. Additionally or alternatively, the one or more computable executable instructions are carried in a signal or carrier wave.

The following provides several non-limiting examples of polypeptide radiotoxicity bio-markers that indicate higher or lower patient radiosensitivity. For these examples, blood serum samples of twenty-three (23) ectomized prostate cancer patients with high and low grade bowel and urinary toxicity (as determined via EPIC or otherwise) were collected before (0 Gy) during (20-26 Gy, 40-46 Gy and 60-66 Gy) and two months after RT. The serum samples were analyzed according to the examples below, and patterns in form of a set of polypeptide M/Z values were identified. Some patient samples were analyzed in four replicates in order to assess the reproducibility which was found to be high enough for reliable classification of the small training set.

It is known that spectra collected on different mass spectrometers differ slightly, e.g. due to imperfections in calibration. It is also known that the same mass spectral peak identified in different subjects may present itself at slightly different M/Z values. Such differences can be due to variation at various levels, including the genetic level and the post-translational modification level. Also, the mass spectrometer has limited mass resolution. As such, each peak or mass is defined as an interval. For estimating an acceptable mass range for a peak definition the M/Z interval is set to ±0.2% of the mean mass of the peak group.

With one example, 10 μL Serum from prostate cancer patients were prepared on CM10 arrays and analyzed according to the following:

### 1. Denaturation

Add 30 μL denaturing buffer U9 (9M urea, 2% CHAPS, 10 mM TRIS, pH 9.0, stored at −80° C.) into the appropriate wells of a 96-well plate.
Pipette 10 μL samples for a concentration of 10%.
Store the plate on ice. Vortex for 20 min at 4° C.
   2. Equilibrate arrays in bioprocessor
Add 100 μl binding buffer (100 mM NH4Ac, 0.2% NP40, pH: 4.5) to each well. Check each well to ensure no bubbles are present.
Incubate for 5 min on shaker (600 rpm).
Remove the buffer by pouring out and tapping bioprocessor on paper towel pile.
Repeat once.
Proceed without drying chip spots.
   3. Dilution of samples, and sample incubation
Dilute denatured samples by adding 60 μL binding buffer to the wells. Immediately pipette the samples into the bioprocessor.
Incubate on plate shaker for 45 min (600 rpm).
Remove samples by pouring out and tapping bioprocessor on paper towel pile.
   4. Washing steps
3× 100 μl binding buffer for 5 min (600 rpm). Discard buffer.
2× 100 μl of washing buffer (5 mM HEPES pH7) for only ca 5 s. Discard buffer.
Remove the bioprocessor and let chips air dry flat on bench.
   5. Matrix preparation (during chip drying)
Centrifuge the tube with matrix powder (ca 15 kg, 2 min)
Prepare fresh 1% TFA (50 μl TFA and 5 ml water)
Add 125 μl ACN and 125 μl 1% TFA to the SPA tube
Vortex for 1 min
Mix it with Eppendorf shaker, 14000 rpm, 15 min
Centrifuge (ca 15 kg, 3 min) to sediment undissolved matrix
Transfer supernatant to a new tube
   6. Matrix addition
2×1 μl SPA (let it dry for 10 min between SPA additions).
   7. Spectrum acquisition & analysis
The arrays were analyzed in a SELDI-TOF MS PCS4000 with settings optimized for the low mass range (peptide range):
Set Mass Range from 2000 to 35000 Da
Set Focus Mass to 8000 Da
Set Matrix Attenuation to 1000 Da
Set Sampling Rate to 400 MHz
Set data acquisition method to SELDI Quantization
Set 1 warming shot with an Energy of 3080 nJ and do not include warming shots after spectrum acquisition.
Set 15 data shots with an Energy of 2800 nJ
Measure partitions 1 of 5
   8. Post acquisition analysis
In the first Pass Peaks with SNR>5 and a valley depth of 0.3 were automatically detected.
The Min Peak Threshold was set to 15.0% of all spectra.
All first Pass Peaks were preserved.
The Cluster mass window was set to 0.2% of mass
In the second Pass Peaks with SNR>2 and a valley depth of 2 were automatically detected.
Estimated Peaks were added to complete Clusters at auto centroid.

With another example, 20 μL of these Serum samples were prepared and analyzed on IMAC30 arrays and analyzed according to the following:

### 1. Denaturation

Add 30 μL denaturing buffer U9 into the appropriate wells of a 96-well plate.
Pipette 20 μL samples into the wells of the plate for samples with a concentration of 20%.
Vortex for 20 min, 4° C., 600 rpm (Thermo Mixer).
   2. Equilibrate arrays in bioprocessor 1
Add 50 μl of 0.1 M copper sulphate (IMAC charging solution) to each well. Check each well to ensure no bubbles are present.
Incubate for 10 min on shaker (600 rpm) at room temperature.
Remove the buffer by pouring out and tapping bioprocessor on paper towel pile.
Repeat once.
Proceed without drying chip spots.
   3. First washing step
Add 200 μl of deionised water to each well. Check each well to ensure no bubbles are present.
Incubate for 1 min on shaker (600 rpm) at room temperature.
Remove the DI water by pouring out and tapping bioprocessor on paper towel pile.
Proceed without drying chip spots.
   4. Equilibrate arrays in bioprocessor 2
Add 200 μl of 0.1 M sodium acetate buffer (IMAC neutralizing solution, pH4) to each well. Check each well to ensure no bubbles are present.
Incubate for 5 min on shaker (600 rpm) at room temperature.
Remove the buffer by pouring out and tapping bioprocessor on paper towel pile.
Proceed without drying chip spots.
   5. Second washing step
Add 200 μl of deionised water to each well. Check each well to ensure no bubbles are present.
Incubate for 1 min on shaker (600 rpm) at room temperature.
Remove the DI water by pouring out and tapping bioprocessor on paper towel pile.
Proceed without drying chip spots.
   6. Equilibrate arrays in bioprocessor 3
Add 200 μl of 0.1 M IMAC binding buffer (0.1M sodium phosphate, 0.5M sodium chloride, pH7) to each well. Check each well to ensure no bubbles are present.
Incubate for 5 min on shaker (600 rpm) at room temperature.
Remove the buffer by pouring out and tapping bioprocessor on paper towel pile.
Repeat once.
Proceed without drying chip spots.
   7. Dilution of samples, and sample incubation
Dilute denatured samples by adding 50 μL binding buffer to the wells. Immediately pipette the samples into the bioprocessor.
Incubate on plate on shaker for 30 min (600 rpm).
Remove samples by pouring out and tapping bioprocessor on paper towel pile.
   8. Last washing steps
2× 200μl IMAC binding buffer for 5 min (600 rpm).
Remove the binding buffer by pouring out and tapping bioprocessor on paper towel pile.
2× 200 μl of deionised water for only ca 5 s (remove immediately).
Remove the bioprocessor and let chips air dry flat on bench for 15-20 min.
   9. Matrix preparation (during chip drying)
Centrifuge the tube with matrix powder (ca 15 kg, 2 min)
Prepare fresh 1% TFA (50 μl TFA and 5 ml water)
Add 125 μl ACN and 125 μl 1% TFA to the SPA tube
Vortex for 1 min
Mix it with Eppendorf shaker, 14000 rpm, 15 min
Centrifuge (ca 15 kg, 3 min) to sediment undissolved matrix
Transfer supernatant to a new tube
   10. Matrix addition
2×1 μl SPA (let it dry for 10 min between SPA additions).
   11. Spectrum acquisition & analysis
The arrays were analyzed in a SELDI-TOF MS PCS4000 with settings optimized for the low mass range (peptide range):
Set Mass Range from 2000 to 35000 Da
Set Focus Mass to 8000 Da
Set Matrix Attenuation to 1000 Da
Set Sampling Rate to 400 MHz
Set data acquisition method to SELDI Quantization
Set 1 warming shot with an Energy of 3520 nJ and do not include warming shots after spectrum acquisition.
Set 15 data shots with an Energy of 3200 nJ
Measure partitions 1 of 5
   12. Post acquisition analysis
In the first Pass Peaks with SNR>5 and a valley depth of 0.3 were automatically detected.
The Min Peak Threshold was set to 15.0% of all spectra.
All first Pass Peaks were preserved.
The Cluster mass window was set to 0.2% of mass
In the second Pass Peaks with SNR>2 and a valley depth of 2 were automatically detected.
Estimated Peaks were added to complete Clusters at auto centroid.

The analyzed mass range includes the mass range of 2000-10000 Da according to p-Value, ROC-Limit, CV and Intensity difference (D). The identified clusters had either a p-Value≦0.06, a ROC-Limit≧0.8 or ≦0.2 or an D≧25 at one time point. Additionally the minimum cluster intensity was set to 1.

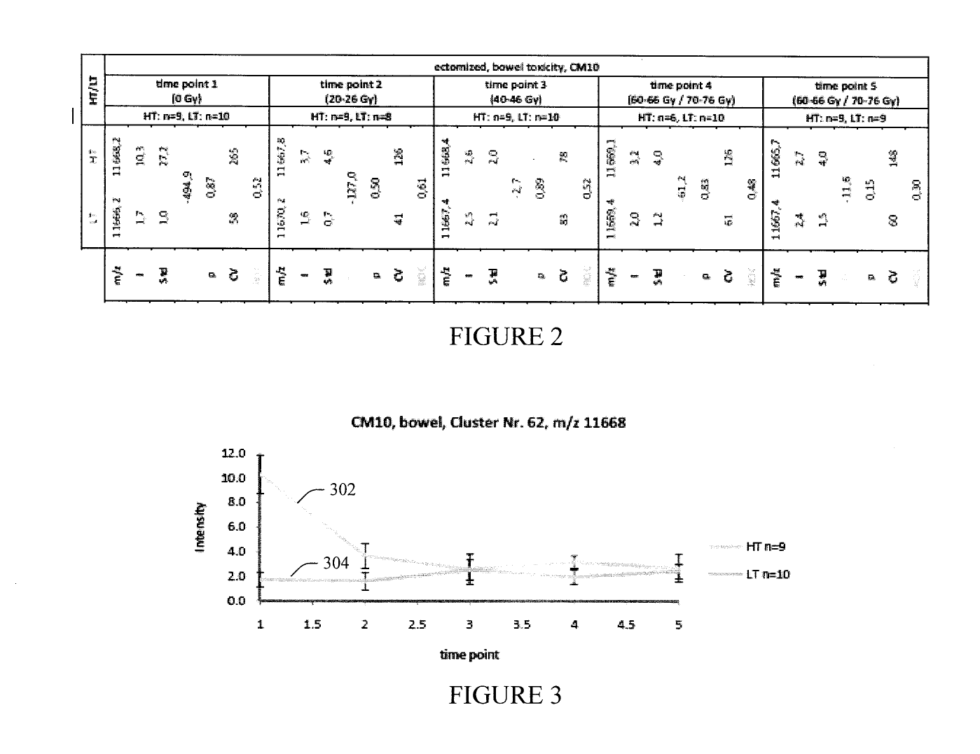

FIG. 2 shows data for a bio-marker having an m/z ratio of 11,668±23 for bowel toxicity found on CM10. In this example, HT represents high toxicity; LT represents low toxicity; m/z represents protein mass in Dalton; I represents mean peak intensity; Std represents standard deviation; D represents Difference of Peak intensity in percent; p represents p-value; CV represents coefficient of variation, and ROC represents Area under ROC curve. This bio-marker has higher intensity differences than standard deviations for high bowel toxicity HT versus low bowel toxicity LT at “time point 1” and “time point 2” on CM10. A high intensity difference at “time point 1” indicates that a radiosensitive patient can be identified before RT. This makes a prognosis of radiotoxicity and individualization of the therapy before starting RT possible. FIG. 3 illustrates intensity curves 302 and 304 for the data of FIG. 2 as a function of time point respectively for the HT and the LT clusters for bowel toxicity with high intensity differences at “time point 1” and “time point 2” found on CM10. Note the high intensity difference (494.9%) at “time point 1,” relative to the other time points.

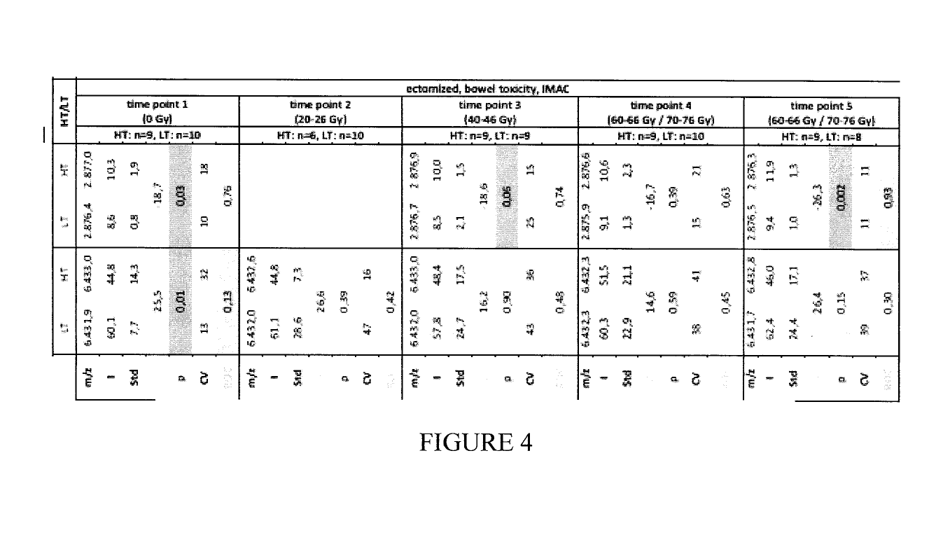

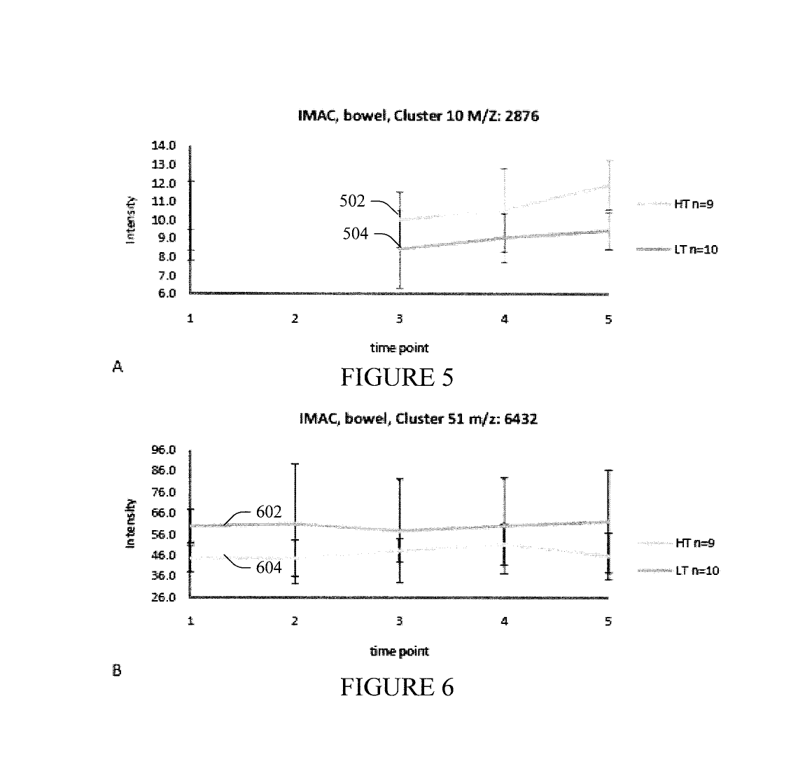

FIG. 4 shows data for bio-markers having m/z ratios of 2,876±6 and 6,432±13 for bowel toxicity found on IMAC. The bio-markers have larger intensity differences than standard deviations for high bowel toxicity versus low bowel toxicity at “time point 5” and “time point 1” on IMAC. Additionally, at these time points the groups can be distinguished with p-values of 0.002 and 0.01 and ROC-Limits of 0.93 and 0.13. FIG. 5 illustrate intensity curves 502 and 504 for the data corresponding to the bio-marker of FIG. 4 having the m/z ratio of 2,876±6 as a function of time point respectively for the HT and the LT clusters for bowel toxicity with high intensity differences at “time point 5” found on IMAC, and FIG. 6 illustrate intensity curves 602 and 604 for the data corresponding to the bio-marker of FIG. 4 having the m/z ratio of 6,432±13 as a function of time point respectively for the HT and the LT clusters for bowel toxicity with high intensity differences at “time point 1” found on PMAC.

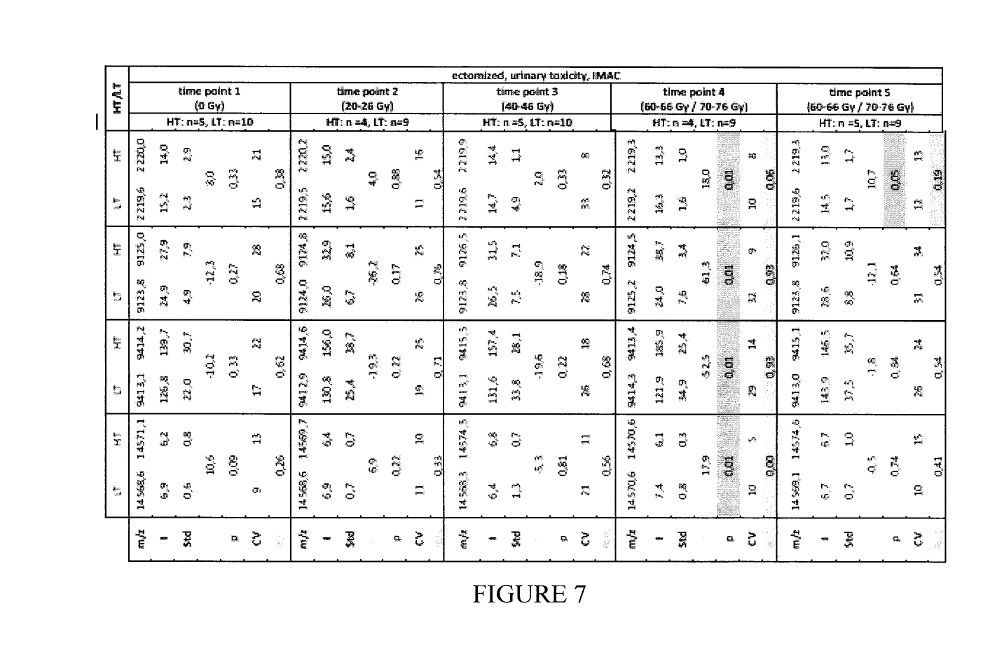

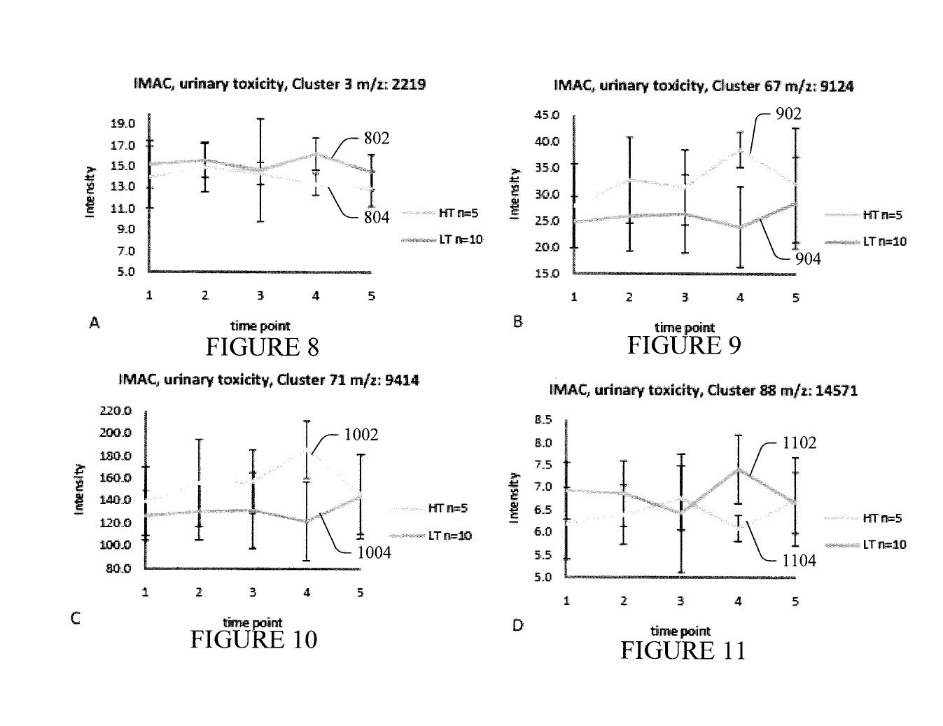

FIG. 7 shows data for bio-markers having m/z ratios of 9,125±18, 2,220±4, 9,414±19 and 14,571±29 for urinary toxicity found on IMAC. The illustrated markers have larger intensity differences than standard deviations for high urinary toxicity versus low urinary toxicity at “time point 4” on IMAC. Additionally, at these time point the groups can be distinguished with p-values of 0.01 and ROC-Limits of 0.00, 0.93, 0.93 and 0.06. FIGS. 8, 9, 10 and 11 illustrate intensity curves 802 and 804, 902 and 904, 1002 and 1004, and 1102 and 1104 respectively for m/z ratios of 9,125±18, 2,220±4, 9,414±19 and 14,571±29 as a function of time point respectively for the HT and the LT clusters for urinary toxicity with high intensity differences at “time point 4” found on IMAC.

Although the above examples are discussed in connection with prostate cancer and bowel and urinary toxicity, it is to be understood that other bio-markers for other cancers (e.g., bladder, rectum, endometrial, cervix, etc.) and/or tissue of interest and/or toxicity of other organs are also contemplated herein.

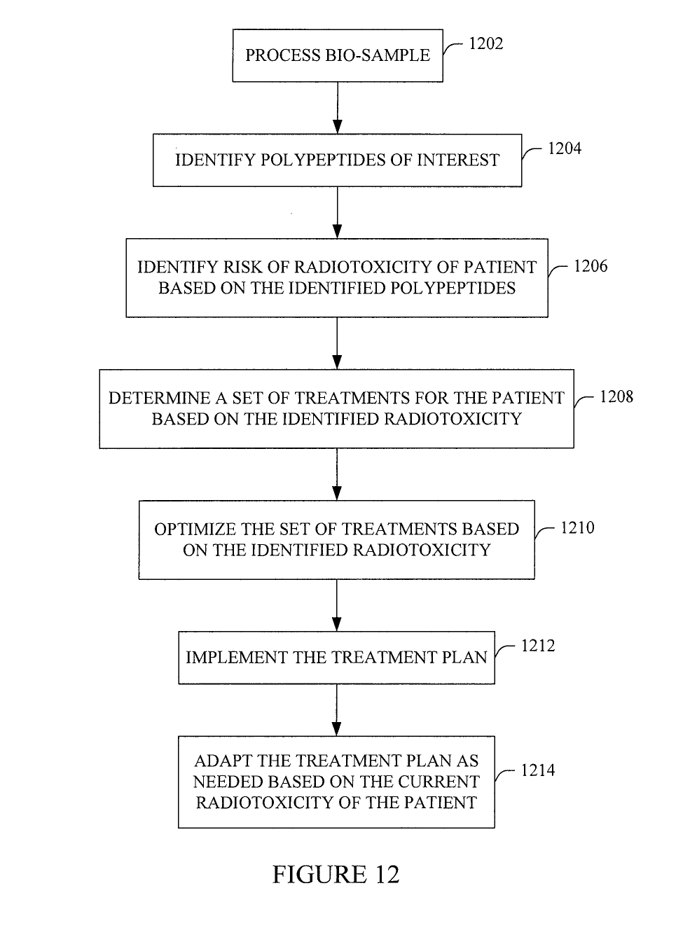

FIG. 12 illustrates a method.

It is to be appreciated that the ordering of the acts in the methods described herein is not limiting. As such, other orderings are contemplated herein. In addition, one or more acts may be omitted and/or one or more additional acts may be included.

At 1202, a bio-sample including polypeptides indicative of a radiotoxicity of a patient is processed and signal indicative thereof is generated. As described herein, the sample can be processed through mass spectrometry, immunoassay, and/or otherwise.

At 1204, a pre-determined set of polypeptide radiotoxicity bio-markers of interest are identified from the polypeptides.

At 1206, a radiotoxicity of the patient is identified based on the pre-determined set of polypeptide radiotoxicity bio-markers. This may include determining radiotoxicity based on intensity peaks before and/or during different time points of radiotherapy treatment for one or more combinations of polypeptides.

At 1208, a set of treatments for a treatment plan of a patient is identified based on the identified radiotoxicity of the patient. This may include determining an initial set of treatments and/or an adapted set of treatments after at least one radiotherapy treatment.

At 1210, the set of treatments is optimized based on the identified radiotoxicity of the patient.

At 1212, the optimized treatment plan is implemented.

At 1214, the treatment plan is adapted, as needed, during implementation based on the current radiotoxicity of the patient.

The above may be implemented via one or more processors executing one or more computer readable instructions encoded or embodied on computer readable storage medium such as physical memory which causes the one or more processors to carry out the various acts and/or other functions and/or acts. Additionally or alternatively, the one or more processors can execute instructions carried by transitory medium such as a signal or carrier wave.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method, comprising:
at least one of creating or adapting a treatment plan for a patient based on a set of serum polypeptides of the patient that are indicative of a radiotoxicity of the patient at least one of before or after at least one of a plurality of radiotherapy treatments of the treatment plan, wherein the radiotoxicity is induced by radiation exposure from the radiotherapy treatment.

2. The method of claim 1, further comprising:
determining masses of polypeptides in a serum sample of the patient;
comparing the determined masses with a pre-determined set of masses of interest;
identifying at least one polypeptide having a mass that satisfies the pre-determined set of masses of interest; and
including only the identified polypeptides in the set of polypeptide radiotoxicity bio-markers,

3. The method of claim 2, further comprising:
determining the masses using mass spectrometry.

4. The method of claim 1, further comprising:
determining masses of polypeptides in a serum sample of the patient using an immunoassay.

5. The method of claim 1, further comprising:
determining peak intensities, concentrations or amounts of the polypeptides in the set of polypeptide radiotoxicity serum markers;
comparing the determined peak intensities with threshold intensities corresponding to higher radiosensitivity and lower sensitivity; and
one of classifying the patient as having higher radiosensitivity in response to a pre-determined combination of the peak intensities mapping to the intensities corresponding to the higher radiosensitivity, or classifying the patient as having lower radiosensitivity in response to the pre-determined combination of the peak intensities mapping to the intensities corresponding to the lower radiosensitivity.

6. The method of claim 5, further comprising:
identifying a sub-set of treatments from a plurality of treatments for the treatment plan based on the classification of the patient.

7. The method of claim 6, wherein the sub-set of treatments includes one or more of external beam radiotherapy, brachytherapy, surgery, chemotherapy, particle therapy, high intensity focused ultrasound, ablation, cryotherapy, watchful waiting or hormonal therapy.

8. The method of claim 6, further comprising:
visually presenting the identified sub-set of treatments; and
including the presented identified sub-set of set of treatments in the treatment plan in response to receiving an input indicative of user acceptance of the presented identified sub-set of treatments.

9. The method of claim 6, further comprising:
automatically including the presented identified sub-set of treatments in the treatment plan.

10. The method of claim 6, wherein the radiotoxicity represents a radiotoxicity after at least one radiotherapy treatment and before at least another radiotherapy treatment, and further comprising:
creating a personalized treatment plan for the patient based on a predicted radiotoxicity of the patient.

11. The method of claim 6, wherein the radiotoxicity represents a radiotoxicity after at least one radiotherapy treatment, and further comprising:
adapting the treatment plan to personalize the treatment plan for the patient based on a current radiotoxicity of the patient.

12. The method of claim 6, further comprising:
optimizing treatment parameters for one or more treatments of the treatment plan based on the polypeptide radiotoxicity bio-markers.

13. The method of claim 12, further comprising:
adding an extra dose boost to a target volume of a radiotherapy treatment of the treatment plan of an individual patient who has lower radiosensitivity.

14. The method claim 12, further comprising:
leaving out an extra dose boost to the target volume of the radiotherapy treatment of the treatment plan of a patient with higher radio sensitivity.

15. The method claim 12, further comprising:
at least one of increasing a predetermined maximum dose of tissue of interest in response to a low predicted or measured toxicity of the tissue of interest or decreasing a predetermined maximum dose of tissue of interest in response to a high predicted or measured toxicity of the tissue of interest.

16. The method claim 12, further comprising:
modifying a dose distribution contour based on the predicted or measured toxicity of the tissue of interest.

17. The method of claim 15, wherein the tissue of interest includes at least one of the urethra, bladder, bowel, or rectum.

18. The method of claim 1, wherein the polypeptide masses of the set of polypeptide radiotoxicity bio-markers include masses from a group consisting of 11,868±23 Da, 2,876±6 Da, 6,432±13 Da, 9,125±18 Da, 2,220±4 Da, 9,414±19 Da and 14,571±29 Da.

19. A system, comprising;
a treatment planning device that facilitates at least one of creating or adapting a treatment plan for a patient based on amounts or concentrations of a set of serum polypeptides of the patient that indicate a high risk of or an early radiotoxicity of the patient to radiation from radiotherapy.

20. The system of claim 19, the treatment planning device, comprising:
a treatment identifier that identifies a set of treatments for the treatment plan based on the set of serum polypeptides.

21. The system of claim 20, wherein the set of treatments are identified before a radiotherapy treatment based on a predicted radiotoxicity of the patient based on the set of serum polypeptides.

22. The system of claim 20, wherein the set of serum polypeptides includes polypeptides with masses from a group consisting of 11,668±23 Da, 2,876±6 Da, 6,432±13 Da, 9,125±18 Da, 2,220±4 Da, 9,414±19 Da and 14,571±29 Da.

23. The system of claim 20, wherein the set of treatments are identified after at least one radiotherapy treatment based on a monitored radiotoxicity of the patient based on the set of serum polypeptides.

24. The system of claim 19, wherein the treatment planning device conveys the treatment plan to a therapy treatment system which automatically loads the treatment plan into the therapy treatment system.

25. The system of claim 19, wherein the treatment planning device visually presents the identified set of treatments.

26. The system of claim 25, wherein a risk of radiotoxicity is visually highlighted in the visually presented information.

27. The system of claim 25, wherein a visual presentation includes a risk toxicity index for the patient.

28. The system of claim 25, the treatment planning device, comprising:
an optimizer that optimizes treatment parameters of treatments in the treatment plan.

29. The system of claim 20, wherein the treatment planning device additionally utilizes one or more of imaging data, non-imaging data, and simulation data to create or adapt the treatment plan.

30. A computer readable storage medium encoded with computer readable instructions, which, when executed by a processor of a computing system, causes the system to: receive information about a polypeptide of a patient that indicates a radiotoxicity of the patient to radiotherapy treatment and create or adapt a treatment plan for the patient based on the received information, wherein the information includes at least a mass of the polypeptide and an intensity peak of the polypeptide.
